(19) United States
(12) Patent Application Publication
(10) Pub. No.: US 2021/0027870 A1
(43) Pub. Date: Jan. 28, 2021

(54) ELECTRONIC HEALTHCARE PLATFORM THAT PROVIDES PERSONALIZED RECOMMENDATIONS FOR PERSONAL CARE PRODUCTS AND HEALTHCARE SERVICES

(71) Applicant: CareNexis, LLC, Cary, NC (US)

(72) Inventor: Daniel West, Cary, NC (US)

(21) Appl. No.: 17/036,022

(22) Filed: Sep. 29, 2020

Related U.S. Application Data
(63) Continuation of application No. PCT/US19/24785, filed on Mar. 29, 2019.
(60) Provisional application No. 62/650,484, filed on Mar. 30, 2018.

(51) Int. Cl. G16H 10/60; G06N 20/00; G06K 9/62; G16H 20/00

(52) U.S. Cl. CPC G16H 10/60; G06N 20/00; G16H 20/00; G06K 9/6267; G06K 9/6215; G06K 9/6218

(57) ABSTRACT

The subject matter described herein includes methods and systems for an electronic healthcare platform that enables users to locate relevant medical or health-related content (e.g., information, products, and services) aggregated from internal and external content sources. In particular, the electronic healthcare platform provides a record database that creates concepts and associations between pieces of content using medical codes and content analytics. The electronic healthcare platform further provides a personalized set of recommendations for personal care products and healthcare services that are directly relevant to customers.

### PRIORITY

This application claims priority to PCT Patent Application No. PCT/US19/24785 filed on Mar. 29, 2019, entitled “ELECTRONIC HEALTHCARE PLATFORM THAT PROVIDES PERSONALIZED RECOMMENDATIONS FOR PERSONAL CARE PRODUCTS AND HEALTHCARE SERVICES,” which claims priority to U.S. Provisional Patent Application No. 62/650,484 filed on Mar. 30, 2018 entitled “ELECTRONIC HEALTHCARE PLATFORM THAT PROVIDES PERSONALIZED RECOMMENDATIONS FOR PERSONAL CARE PRODUCTS AND HEALTHCARE SERVICES,” the entire contents of which are incorporated by reference herein.

### TECHNICAL FIELD

The presently disclosed subject matter relates to an electronic healthcare platform that aggregates healthcare content from multiple sources and analyzes the content to determine concepts and related associations between the content. In particular, the electronic healthcare platform provides a database that creates and stores associations between various types of healthcare content from multiple content sources based on medical codes to provide personalized recommendations for specific products and services that are tailored to the specific needs of the users of the platform.

### BACKGROUND

Medical care has become increasingly complex and continues to grow even more complex. As technology advances, patients increasingly would like to able to manage various aspects of their own treatment.

Patients now have access to more information than ever before. For example, electronic health records (EHRs), which are an electronic version of a patient's paper chart, have become commonplace in the industry. In addition, patients now have access to vast amount of information resources available on the internet. Similarly, patients are now able to research and purchase medical products, such as over-the-counter products and prescription products, more easily than ever before.

Patients and/or other people can learn about medical conditions, ailments, and/or symptoms in a number of ways. For example, a person who is feeling sick may type their symptoms into an internet search engine, such as Google. The search engine may provide a number of results of articles relating to their symptoms. These articles often come from reputable medical-information websites, such as WebMD; the Center for Disease Control's (CDC) website; the Mayo Clinic's website; MedicineNet.com; or the National Institute of Health's (NIH) MedlinePlus website.

Similarly, a person looking for products relating to medical conditions, ailments, and/or symptoms may type their condition/ailment/symptoms into an internet search engine (such as Google) or an online marketplace (such as Amazon). For example, a person suffering from urinary incontinence may search the internet for urinary incontinence products to research and ultimately buy any products they may be looking for. This process, however, can be overwhelming to a user/patient facing a new diagnosis, or to a user/patient who is unfamiliar with all the treatment options and/or products that exist for their medical condition/ailment. In addition, the cost of buying numerous products to figure out which ones work best can be expensive and frustrating to a person suffering from a medical condition/ailment.

Medical codes (also sometimes referred to as diagnostic codes or billing codes) are used in the healthcare industry as a type of shorthand for various diagnoses and treatments. They describe medical conditions/ailments and corresponding treatments. They are also used for billing purposes, such as determining the cost of services, products, and/or medication/drugs. Medical codes may also be used to relate and/or link one disease, ailment, or condition, or medication/drugs to others.

There are numerous types of medical codes, including but not limited to Healthcare Common Procedure Coding System (HCPCS) codes, International Classification of Diseases (ICD) codes, ICF codes for disabilities, diagnosis-related groups (DRG), National Drug Codes (NDC), Code on Dental Procedures and Nomenclature (CDT) codes, DSM-IV-TR codes for psychiatric illnesses. Additionally, the Systematized Nomenclature of Medicine-Clinical Terms (SNOMED CT) also provides a standardized, multilingual vocabulary of clinical terminology, including codes. These medical codes and SNOMED terminology may be used to assist health care professionals with exchanging health information. However, these codes and terminologies can be confusing and ultimately not particularly helpful to a patient who is trying to research their condition at home and/or shop for related products/services at home.

Accordingly, a need exists for an electronic platform that associates medical codes with informational resources, products, services, and/or service providers related to those medical codes.

### SUMMARY

The present invention provides systems, methods, and apparatuses for an electronic healthcare platform that aggregates healthcare content from multiple sources and creates associations between the integrated content using medical codes and other associations.

According to one embodiment of the present invention, a method of providing personalized healthcare content to a user through an electronic healthcare platform is disclosed. The method includes aggregating healthcare content by analyzing the healthcare content to determine a concept relevant to the healthcare content. The healthcare content includes at least one of medical code data, licensed content, original content, product content, and service content. The method further includes creating a concept record associated with the healthcare content based on the analysis of the healthcare content. The concept record includes concept metadata. The concept record further includes a reference to the healthcare content. The method further includes storing the concept record in a record database. The method further includes storing an association record in the record database that links the concept record with another concept record stored in the record database. The association record is created based on information contained in the concept record or information contained in the healthcare content. The method further includes providing the healthcare content in response to a query, wherein the healthcare content is determined to be relevant to the query based on at least one of the concept record and the association record.

In one embodiment of the method of providing personalized healthcare content through an electronic healthcare platform, the association record provides an association between a first concept and a second concept.

In one embodiment of the method of providing personalized healthcare content through an electronic healthcare platform, the association between the first concept and the second concept is determined based on one or more medical codes related to the first concept and one or more medical codes related to the second concept.

In one embodiment of the method of providing personalized healthcare content through an electronic healthcare platform, the concept relevant to the healthcare content is determined based one or more medical codes.

In one embodiment of the method of providing personalized healthcare content through an electronic healthcare platform, the query is received from a front-end interface that includes a branded website that provides access to the aggregated content in the electronic healthcare platform.

In one embodiment of the method of providing personalized healthcare content through an electronic healthcare platform, the query is received from a website that contains an embedded object from the electronic healthcare platform, the website being a third-party website that is not part of the electronic healthcare platform

In one embodiment of the method of providing personalized healthcare content through an electronic healthcare platform, a concept associated with search parameters of the query is used to determine whether the healthcare content is relevant to the query.

In one embodiment of the method of providing personalized healthcare content through an electronic healthcare platform, the analyzing the healthcare content to determine a concept relevant to the healthcare content is performed using a machine-learning technique.

In one embodiment of the method of providing personalized healthcare content through an electronic healthcare platform, the machine-learning technique determines that the concept is relevant to the healthcare content by performing a text-analysis technique to find similarities in the healthcare content.

In one embodiment of the method of providing personalized healthcare content through an electronic healthcare platform, the machine-learning technique determines that the concept is relevant to the healthcare content by performing concept categorization and classification using a clustering technique to find similarities in the healthcare content.

According to another embodiment of the present invention, a system for providing personalized healthcare content is disclosed. The system includes a front-end user interface that includes an embedded component that displays electronic healthcare content. The system further includes a back-end server. The back-end server includes a memory that includes a record database. The back-end server further includes a processor. The processor of the back-end server is configured for aggregating healthcare content by analyzing the healthcare content to determine a concept relevant to the healthcare content. The healthcare content includes at least one of medical code data, licensed content, original content, product content, and service content. The processor of the back-end server is further configured for creating a concept record associated with the healthcare content based on the analysis of the healthcare content. The concept record includes concept metadata. The concept record further includes a reference to the healthcare content. The processor of the back-end server is further configured for storing the concept record in the record database. The processor of the back-end server is further configured for storing an association record in the record database that links the concept record with another concept record stored in the record database. The association record is created based on information contained in the concept record or information contained in the healthcare content. The processor of the back-end server is further configured for providing the healthcare content in response to a query, wherein the healthcare content is determined to be relevant to the query based on at least one of the concept record and the association record.

In one embodiment of the system for providing personalized healthcare content, the association record provides an association between a first concept and a second concept.

In one embodiment of the system for providing personalized healthcare content, the association between the first concept and the second concept is determined based on one or more medical codes related to the first concept and one or more medical codes related to the second concept.

In one embodiment of the system for providing personalized healthcare content, the concept relevant to the healthcare content is determined based one or more medical codes.

In one embodiment of the system for providing personalized healthcare content, the query is received from a branded website that is part of the front-end interface that provides access to the aggregated content in the electronic healthcare platform.

In one embodiment of the system for providing personalized healthcare content, the query is received from a website that contains an embedded object from the electronic healthcare platform, the website being a third-party website that is not part of the electronic healthcare platform.

In one embodiment of the system for providing personalized healthcare content, a concept associated with search parameters of the query is used to determine whether the healthcare content is relevant to the query.

In one embodiment of the system for providing personalized healthcare content, the analyzing the healthcare content to determine a concept relevant to the healthcare content is performed using a machine-learning technique.

In one embodiment of the system for providing personalized healthcare content, the machine-learning technique determines that the concept is relevant to the healthcare content by performing a text-analysis technique to find similarities in the healthcare content.

In one embodiment of the system for providing personalized healthcare content, the machine-learning technique determines that the concept is relevant to the healthcare content by performing concept categorization and classification using a clustering technique to find similarities in the healthcare content.

According to another embodiment of the present invention, a method of associating healthcare content with a concept is disclosed. The method of associating healthcare content with a concept includes analyzing a first piece of healthcare content to determine a concept related to the first piece of healthcare content. The concept related to the first piece of healthcare content is determined based on the first piece of healthcare content. The method of associating healthcare content with a concept further includes associating the first piece of healthcare content with a first concept record. The first concept record includes a concept ID and concept metadata. The method of associating healthcare content with a concept further includes analyzing a second piece of healthcare content to determine a concept related to the second piece of healthcare content. The concept related to the second piece of healthcare content is determined based on the second piece of healthcare content. The method of associating healthcare content with a concept further includes associating the second piece of healthcare content with a second concept record. The second concept record includes a concept ID and concept metadata. The method of associating healthcare content with a concept further includes associating the first concept record with the second concept record by creating an association record containing the concept ID of the first concept record and a concept ID of the second concept record.

In one embodiment of the method of associating healthcare content with a concept, the first concept record includes a reference to the first piece of healthcare content and the second concept record includes a reference to the second piece of healthcare content.

In one embodiment of the method of associating healthcare content with a concept, the concept metadata of the first concept record includes a label relevant to the first piece of healthcare content and the concept metadata of the second concept record includes a label relevant to the second piece of healthcare content.

In one embodiment of the method of associating healthcare content with a concept, the concept metadata of the first concept record includes a medical code relevant to the first piece of healthcare content and the concept metadata of the second concept record includes a medical code relevant to the second piece of healthcare content.

In one embodiment of the method of associating healthcare content with a concept, the association between the first concept record and the second concept record is created based on a comparison of the concept related to the first piece of healthcare content and the concept related to the second piece of healthcare content, wherein the comparison determines a similarity between concept metadata of the first concept record and concept metadata of the second concept.

According to another embodiment of the present invention, a method of providing a personalized healthcare website as part of an electronic healthcare platform is disclosed. The method of providing a personalized healthcare website as part of an electronic healthcare platform includes storing a profile associated with a user of the electronic healthcare platform, wherein the profile includes a medical code associated with the user. The method of providing a personalized healthcare website as part of an electronic healthcare platform further includes providing a recommendation to the user based on the medical code associated with the user. The recommendation includes a product associated with the medical code.

In one embodiment of the method of providing a personalized healthcare website as part of an electronic healthcare platform, the product is associated with the medical code using machine-learning.

In one embodiment of the method of providing a personalized healthcare website as part of an electronic healthcare platform, the recommendation further includes an article associated with the medical code.

In one embodiment of the method of providing a personalized healthcare website as part of an electronic healthcare platform, the recommendation further includes a healthcare service associated with the medical code.

According to another embodiment of the present invention, an electronic healthcare platform is disclosed. The electronic healthcare platform includes a user interface layer. The user interface layer is configured to display content using an embedded object. The electronic healthcare platform further includes a services layer. The services layer includes an API configured to provide healthcare content to the embedded object. The electronic healthcare platform further includes a content layer. The content layer includes a content library containing healthcare content. The electronic healthcare platform uses a code-lock algorithm to associate healthcare content in the content library with a healthcare concept.

In one embodiment of the electronic healthcare platform, the healthcare content provided to the embedded object in the user interface layer through the API in the services layer is selected based on an association between the healthcare content and the healthcare concept.

In one embodiment of the electronic healthcare platform, the code-lock algorithm associates healthcare content in the content library with the healthcare concept using a medical code.

In one embodiment of the electronic healthcare platform, the code-lock algorithm associates healthcare content in the content library with the healthcare concept using a medical condition.

In one embodiment of the electronic healthcare platform, information relating to the healthcare concept is stored as a concept record in a record database.

In one embodiment of the electronic healthcare platform, the concept record is linked to a related concept record in the record database using an association record stored in the record database.

In one embodiment of the electronic healthcare platform, the embedded object is an HTML object associated with the healthcare concept using a concept ID.

In one embodiment of the electronic healthcare platform, the content layer is configured to aggregate healthcare content from a content source by storing a reference to the content source in the record database.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE DRAWINGS

FIG. 1 depicts an exemplary embodiment of the electronic healthcare platform.

FIG. 2 depicts an exemplary embodiment of the software architecture of the electronic healthcare platform.

FIG. 3 depicts an exemplary method of providing content to a user of the electronic healthcare platform.

FIG. 4 depicts an exemplary structure of a concept record stored in the record database of the electronic healthcare platform.

FIG. 5 depicts a relationship between different types of content and/or concepts within the platform and medical codes used within the platform.

FIG. 6 depicts associations between various types of source content within the platform.

FIG. 7 depicts associations between various types of source content within the platform along with the platform's content service providing an added model of concepts and associations that augment the original source content.

FIG. 8 depicts associations between various types of source content within the platform as well as the platform's content service augmenting the source content with concepts and associations within the platform.

FIG. 9 depicts an exemplary process flow for a user of the electronic healthcare platform to purchase a recommended product.

FIG. 10 depicts an exemplary process flow for finding relevant medical content within the electronic healthcare platform.

### DETAILED DESCRIPTION

The following description and figures are illustrative and are not to be construed as limiting. Numerous specific details are described to provide a thorough understanding of the disclosure. However, in certain instances, well-known or conventional details are not described in order to avoid obscuring the description. References to “one embodiment” or “an embodiment” in the present disclosure can be, but not necessarily are, references to the same embodiment and such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The presently disclosed subject matter relates to an electronic healthcare platform that enables users to find, try, and buy specific products and/or services that are associated with health condition data, such as medical codes and/or natural language searches. The presently disclosed subject matter allows for advanced linking and cross-referencing between one or more medical codes and one or more informational resources, products, and/or services by creating concepts and associations based on the content using machine learning, artificial intelligence, and analytics.

The electronic healthcare platform described herein provides one or more of the following features/functionalities: (1) shopping cart and e-commerce capability (including the ability to order/request product samples); (2) advanced information-searching capability; (3) capability to store and/or manage profile information for one or more users; (4) capability to store and/or manage patient information for users; (5) capability to store and/or manage Care Plan information for users; (6) capability to connect with a trusted professional; (7) capability to research medical conditions/ailments; and (8) capability to identify relationships between medical conditions/ailments/symptoms and products/services using artificial intelligence and/or machine learning.

The electronic healthcare platform may access, analyze, and compile both licensed content and original content relating to health care information. The electronic healthcare platform may be integrated with third-party websites or other information services, such as services provided by one or more corporate partners. Content may include medical products and services that are related to various medical conditions.

When the electronic healthcare platform aggregates source content into the platform, the platform augments the source content with concepts and associations that are stored in a database. Content comprises concepts and associations between concepts. Each content source includes a model or representation of concepts and a model or representation of associations. The platform defines a model of concepts and associations that augments the original source content. The platform creates the concepts and associations using machine-learning or other methods of association (e.g., manual association). When the content has been augmented with additional concepts and associations, those concepts and associations are used to provide additional relevant content to the users through the platform (e.g., articles, products, and services).

The electronic healthcare platform may allow a user to search for products and/or services using medical codes or keyword searches, which may be performed through the various front-end interfaces. The electronic healthcare platform may also allow a user to search for information using keywords, ailments, symptoms, and/or medical codes. For example, a user may search for ICD-10-CM>R32 and/or ICD-10-CM>N39.41. Such a search may return results for “urinary incontinence.” The electronic healthcare platform may allow for natural-language search of the database. For example, a user may be able to search for “I accidentally wet my pants,” “I urinate when I cough,” or “frequent bathroom trips.” Those searches may return results for “urinary incontinence”.

The electronic healthcare platform may associate products, services, and/or service providers with medical codes and keywords, and it may return those associated products and/or services when a corresponding search is run. The electronic healthcare platform may also associate informational resources, such as articles and videos, with medical codes and keywords. This association may be performed manually, automatically, or through machine learning and/or artificial intelligence.

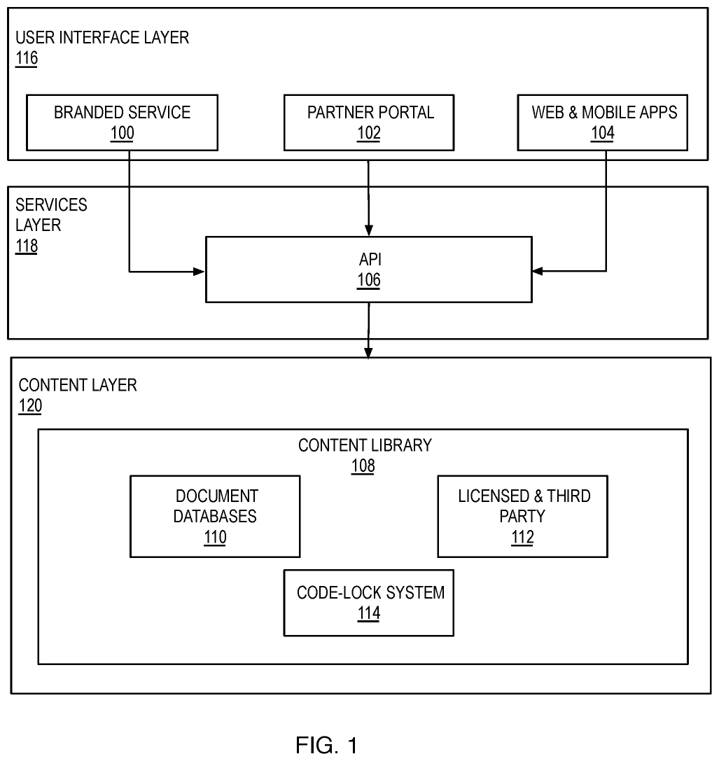

FIG. 1 depicts an exemplary embodiment of the electronic healthcare platform.

Referring to FIG. 1, the system architecture for the electronic healthcare platform comprises three layers: (1) a user interface layer 116, which may be implemented as a front-end system; (2) a services layer 118, which may be implemented between the front-end system and the back-end system; and (3) a content layer 120, which may be implemented as a back-end system.

The user interface layer 116 provides one or more access points for users of the electronic healthcare platform to access the platform. The access points provided by the user interface layer 116 includes a branded service 100, one or more partner portals 102, and/or web and mobile apps 104. The branded service 100 of the user interface layer 116 may include a CarePlanner website, which is a website hosted as part of the electronic healthcare platform (described in more detail below). The partner portals 102 are third-party websites that may have a partnership with the electronic healthcare platform and contain embedded components in the third-party websites that display platform content and provide a link to the platform. The web and mobile apps 104 include mobile applications and/or web-based applications that provide platform content and access to the platform. Each of these elements of the user interface layer 116 includes one or more embeddable components for including and/or integrating resources from the platform into web-based applications (e.g., access through a web browser) and/or mobile applications (e.g., Apple iOS apps, Android apps). In one embodiment, the user interfaces layer is implemented using a Javascript SDK.

The user interface layer 116 may provide a search functionality that allows a user (i.e., person on the internet, patient, healthcare professional) to research medical topics by searching using medical codes, keywords, ailments, symptoms, and/or conditions. The electronic healthcare platform may query the platform's record database to locate informational resources, products, and/or services associated with the medical codes, keywords, ailments, symptoms, and/or conditions input by the user.

Similarly, the user interface layer 116 may provide a list of common conditions that the user can browse to get more information. The list of common conditions may change based on data gathered and analyzed by the electronic healthcare platform. For example, during flu season, the flu may be listed as a common condition, but during summer, swimmer's ear may be listed as a common condition.

The user interface layer 116 may provide the user with a symptom questionnaire. The symptom questionnaire may include a list of questions used to help a patient determine medical conditions/ailments that may be relevant to them. The electronic healthcare platform may use the user's answers to the symptom questionnaire to query the back-end system to find information and/or products associated with the user's relevant medical conditions/ailments.

When a user selects a medical condition/ailment through the user interface layer 116, either by browsing conditions, or by keyword searching, or by medical code searching, the platform provides informational resources relating to that medical condition/ailment to the user. The informational resources for a particular medical condition/ailment may include a brief description, key care conditions, solutions, community, media library, educational videos, the ability to speak to a medical team member, and licensed content from a third-party content provider.

The services layer 118 provides services to the user interface layer 116 from the content layer 120. The services layer 118 may include one or more APIs (application programming interfaces) 106 that are used to access the services provided by the services layer and that manage one or more embedded components, dynamic components, and/or RESTful content resources within the layers of the electronic healthcare platform.

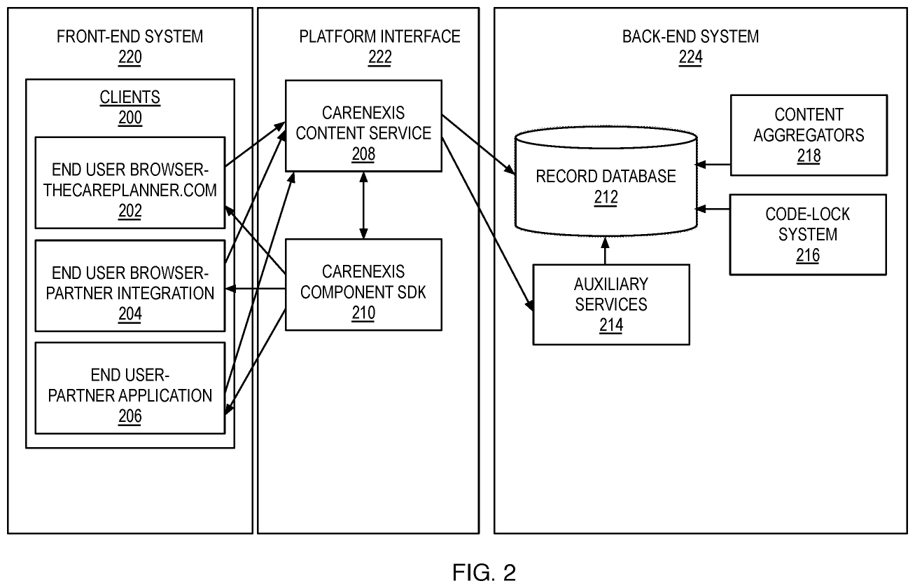

The services layer 118 may provide services such as aggregating content, searching content, filtering content, and performing analytics within the platform. The service for aggregating content may allow the platform to aggregate content from various content sources, including content sources within the platform as well as content sources external to the platform. The content aggregation may be performed using artificial intelligence and/or machine learning to categorize and/or classify the content as it is added to the platform (explained in more detail in the context of FIG. 2, below). The service for searching content may be performed using natural language search, keyword search, and/or search by medical codes. The service for filtering content may be performed by filtering based on concepts and/or associations between content or by filtering based on one or more medical codes. In one embodiment, the services layer 118 is implemented using a RESTful service-oriented architecture, which may allow for interoperability between computers using and/or accessing the platform. The RESTful service oriented architecture allows the computers that are using/accessing the platform to access and manipulate the content of the platform through a uniform and predefined set of stateless operations.

The content layer 120 provides a flexible, scalable, high-performance content architecture that allows the platform to aggregate, store, and/or access content that is within the platform and/or content that is accessible through the platform. The content layer 120 may include a content library 108, document databases 110, licensed and third-party content 112, and a code-lock system 114. The content layer 120 aggregates all the platform's licensed content source into a single seamless interface, so that content from various sources can be accessed through the platform without regard to the source of the content. The content included in the platform may include original content created by the platform, as well as content from partner brands. It may further include licensed content from third parties. It may further include curated content from third parties. The content in the content layer 120 may include information and/or data relating to medical codes (e.g., ICD9, ICD10, CPT, SNOMED CT, HSPC, and NUCC medical code systems), medical conditions, wellness resources, medical treatments, medical tests, diet and nutrition resources, lifestyle resources, and/or pharmaceutical resources.

In one embodiment, the content layer 120 may be configured to organize aggregated content in a hierarchical fashion such that different content levels are used. For example, content in the content layer may be categorized by Parent Topic at the highest level of the hierarchy, then Child Topic at the next level of the hierarchy, and Medical Code at the next level of the hierarchy (e.g., Diabetes->Diabetes Type 1 in Children->E10.29).

In one embodiment, the content layer 120 may be implemented using one or more NoSQL record databases. In other embodiments, the content layer may be implemented using other databases that are known in the art, including distributed ledgers (e.g., blockchain). The databases may be located in one physical location or spread over multiple databases located remote from one another. As explained in more detail below, the content layer uses machine learning and/or artificial intelligence to discover, organize, and/or select relevant, personalized content for a user.

FIG. 2 depicts an exemplary embodiment of the software architecture of the electronic healthcare platform.

Referring to FIG. 2, the software architecture of the electronic healthcare platform includes various type of software interfaces within the front-end system 220, which comprises one or more clients 200.

The front-end system 220 may include an online website accessible through an internet web browser, for example, on a desktop or laptop computer or on a mobile device, such as a smartphone or tablet, or through an application (“app”) on a mobile device, such as a smartphone or tablet. The website may be open and accessible to the general public. In addition to being open to the general public, the website may also allow users to create a profile and save their relevant information as part of their profile. For example, users may enter their personal information, such as name, address, contract information, gender, height, weight, etc., and any relevant medical issues. The clients 200 may include, for example, a CarePlanner website 202, which can be accessed by a user through an internet browser (e.g., Google Chrome, Microsoft Internet Explorer, Apple Safari, or Mozilla Firefox) or a CarePlanner mobile application. The clients 200 may further include partner websites (e.g., licensed content providers such as Healthwise) or partner portals (e.g., a healthcare provider's EHR system), which can be accessed by a user through an internet browser. With partner websites and/or partner portals, the platform content may be integrated into the partner websites such that the platform content is embedded in existing partner web pages such that the platform content can be accessed through the partner websites. The clients 200 may further include a mobile application for accessing the platform (e.g., Android app, Apple iOS app, Microsoft Windows app, etc.). In one embodiment, the front-end system may include a CarePlanner website, a Care Directory website, and/or a Care Directory Subscriber Interface.

The software architecture of the electronic healthcare platform includes a platform interface 220. The platform interface 220 is where, within the platform, the publicly accessible components are isolated. The platform interface 220 includes a content service 208 and a component SDK 210. The content service 208 is a publicly accessible web service for accessing and querying content through the platform (e.g., web APIs for accessing content within the platform). Content within the platform may be aggregated from multiple sources. As used herein, content includes any healthcare-related information, such as articles, medical codes, medical products, medical services, etc. The content sources may include, for example, licensed content from third-parties, public standards and/or conventions (e.g., medical codes), original content authored and/or owned by the platform, original content authored by independent sources (e.g., bloggers, etc.) owned and/or curated by the platform.

The platform's content service 208 provides a unified access point and model for all content. The model maps the original source content (e.g., licensed content hosted by a third-party website) into the platform's content model. By mapping the original source content into the platform's content model, the platform may define new and/or additional associations between concepts in the content that do not already exist in the original source models. In addition, the platform's content model allows for the platform's web components to be designed to use the platform's content model.

The platform's web component SDK 210 is a publicly distributed resource to embed platform content into third-party web pages with minimal effort or impact on those pages into which the content is being integrated. The web component SDK 210 comprises a bundled library of page elements that can render one or more of the platform's concepts. For example, the web component SDK 210 provides objects that can be embedded into a third-party website to display platform content alongside the third-party website's content. The components may be configured to render a single concept or multiple concepts. Each component within platform may be configured with one or more of the platform's concept IDs.

The web component SDK 210 provides common HTML page elements based on the platform's concept abstraction. The page elements provided by the component SDK may include specialized elements for specific data types (e.g., a Shopify storefront). The element selection in the component SDK may be dynamic, such that different elements may be selected at render time based on, for example, details of the concept being displayed or requirements of the page on which the concept is being displayed. The component SDK may be embodied in multiple forms, such as web components and/or HTML.

As one example, the web component SDK 210 may include a “Banner” page element, which can render platform content on a web page as a banner element. Clicking on the banner element from the web page takes the user of the web page to the platform so that the user can navigate the platform for additional content related to the banner content though one or more associations.

As another example, the web component SDK may include an “Article” page element, which can render platform content on a web page as an article. Although not required, generally the content being rendered for an article page element is an article. Clicking on the article element from the web page takes the user of the web page to the platform so that the user can navigate the platform for additional content related to the article content though one or more concepts and/or associations.

As another example, the web component SDK may include a “Product Tile” page element, which can render platform content on a web page as a tile for a product. The product tile may include information about a product that can be purchased or requested as a sample through the platform. Clicking on the product tile element from the web page takes the user of the web page to the platform so that the user can buy or request a sample of the product and/or navigate the platform for additional content related to the product through one or more concepts and/or associations.

The banner, article, and product tile page elements discussed above are used for rendering one of the platform's concepts. The web component SDK may also include page elements that can render more than one of the platform's concepts at the same time. These multiple-concept page elements may be used to render concepts that are related to one another through one or more associations within the platform.

For example, the web component SDK may include a “Carousel” page element, which may be configured to render a collection of other web components, as described above. The carousel page element may render multiple pieces of platform content on a web page as a scrollable element. The carousel may include multiple product tiles, with each product tile including information about a product that can be purchased or requested as a sample through the platform, where each product tile in the carousel displays a different product associated with a particular concept (e.g., medical code). The multiple product tiles of the carousel may be clicked through using an arrow or other method of browsing through the various product tiles. Clicking the active product tile in the carousel element from the web page takes the user of the web page to the platform so that the user can buy or request a sample of the product and/or navigate the platform for additional content related to the product through one or more associations.

As another example, the web component SDK may include an “Accordion” page element, which may be configured to render a collection of other web components, as described above. The accordion page element may render multiple pieces of platform content on a web page as an expandable accordion page. The accordion may include multiple pieces of platform content.

As another example, the web component SDK may include a “Tabs” page element, which may be configured to render a collection of other web components, as described above. The Tabs page element may render multiple pieces of platform content on a web page as a tabbable page with multiple pieces of platform content on a web page. The Tab element may include multiple pieces of platform content.

The platform's library of page elements may be imported into a third-party web page as a script by, for example, embedding HTML in the web page that calls the various platform components. Each component within platform is configured with one or more concept IDs, which the platform uses to display relevant content in the component. The concept rendered in a component may be changed via script.

The software architecture of the electronic healthcare platform includes a back-end system 224. The back-end system 224 includes a record database 212, auxiliary services 214, content aggregators 218, and a code-lock system 216.

The record database 212 stores concept records and associations between concepts for content that has been aggregated by the platform (e.g., licensed content, platform original content, curated content, etc.). The record database 212 contains medical codes, SNOMED terminology and/or codes, and keywords for known medical conditions/ailments and symptoms. The keywords for known medical/conditions/ailments and symptoms may include accepted medical terminology as well as commonly used natural-language keywords. For example, the record database may contain terms “urinate,” “urinary incontinence,” “bed wetting,” and “accidentally wet my pants,” and those terms may be associated with one another using one or more concepts and/or associations.

The record database 212 may store multiple layers of medical codes. The database may store codes from various coding systems, such as, for example, specific ICD-9 and ICD-10 codes, SNOMED CT codes, HSPC codes, NUCC medical codes, and/or Current Procedural Terminology (CPT) codes.

The record database 212 may be populated in a number of ways. As one example, the database may be populated manually. A database administrator may log in to the electronic healthcare platform and manually code information in the database. This manual coding may include manually creating cross-references between medical codes, keywords, products, services, and/or information resources using accepted medical knowledge. As another example, the database may be populated by ingesting information from third-party content providers and/or third-party online shopping portals (using content integrators 218, as explained in more detail below).

The record database 212 may further contain links to articles, information, products, services, and service providers that may be relevant to one or more medical conditions. The record database 212 may associate the medical codes, SNOMED terminology, and/or keywords with the relevant content within the platform (e.g., products, services, and/or service providers).

The record database 212 may further contain links and/or cross-references that associate the medical codes (e.g., ICD-9 and ICD-10 codes), SNOMED terminology and codes, and/or keywords with content within the platform (e.g., articles, information, products, services, and/or service providers), as well as with other medical codes, SNOMED terminology, and/or keywords.

The record database 212 may further contain RX-normative data (e.g., medication data). The record database 212 may create additional cross-references and links involving the medication data.

The record database 212 may further include fields with additional information, such as, for example, age statistics of patients and purchasers, geographical location of patients and purchasers, percentage of population, predicted success rate as correlated to specific medical codes, etc.

The record database 212 may receive EHR data from an EHR system. Alternatively, records from the record database 212 may be provided to an EHR system through organic means or through searching, from the patient portal, and from other traffic drivers.

The record database 212 may be implemented as a NoSQL database, or it may be implemented using, for example, using Ruby on Rails®, PostgresQL®, ActiveAdmin®, and/or GraphQL®.

The auxiliary services 214 include additional services that may be added to the platform to add or enhance features. For example, the auxiliary services 214 may include search optimization and query optimization.

The content aggregators 218 include source-specific logic for mapping the source content onto the platform's content model. The content aggregators 218 are loosely coupled with the electronic healthcare platform, such that the content aggregators 218 can be updated with minimal impact on the electronic healthcare platform. Each supported content source that is aggregated into the platform includes a content integrator. The content aggregators 218 are configured to aggregate content from one or more content sources into the electronic healthcare platform or to link one or more content sources to the electronic healthcare platform. As shown in FIG. 2, the content sources may include licensed content providers, one or more medical codes systems, original platform content, e-commerce data, inventory management and analytics data, and website data.

The content aggregators 218 may import licensed content into the record database 212. For example, Healthwise is a content provider of health information content and relevant medical codes. The content aggregators 218 may import Healthwise content into the record database 212, import information relating to the Healthwise content into the record database 212, and/or link the Healthwise content from concept records stored in the record database 212.

Similarly, the National Institute of Health's (NIH) U.S. National Library of Medicine includes a Unified Medical Language System (UMLS). The UMLS contains vast amounts of data relating to medical conditions. The content aggregators 218 may access information in the UMLS to import and/or link additional content into record database 212. Other similar health information content providers may include the AARP and the National Institute of Health's National Library of Medicine.

The content aggregators 218 may further import medical codes into the record database 212. As explained above, the electronic healthcare platform may be configured to support the ICD9, ICD10, CPT, SNOMED CT, HSPC, and NUCC medical code systems. The content aggregators 218 import medical codes from each of these medical code systems into the record database 212.

The content aggregators 218 may further import original content from the platform into the record database 212. The platform may include original content, such as articles, questionnaires, etc. that are created by experts associated with the platform.

The electronic healthcare platform may include other functionality implemented in the back-end system 224 that can be accessed through the front-end system 220 and/or the platform interface 222.

For example, the electronic healthcare platform may include an e-commerce component, such as, for example, Shopify®. The e-commerce component may include checkout, payment, order, and fulfillment functionality for product purchases. The content aggregators 218 may aggregate product and other e-commerce data into the record database 212, such as products offered for sale, user profiles, user purchase history, etc. The e-commerce system (e.g., Shopify®) allows for real-time purchases (as well as requests for free samples) of products and/or services that are found using the electronic healthcare platform. The e-commerce component may be communicatively coupled to the front-end system 220 and provide cart/checkout services to the front-end system 220, as well as provide product data to the front-end system 220. E-commerce systems are well-known in the art and are not described in detail here.

As another example, the electronic healthcare platform may include an inventory management and analytics component, such as, for example, Salsify®. The content aggregators 218 may aggregate inventory data into the record database 212, such as data relating to inventory of products available for purchase through the platform. The front-end system 220 may also be communicatively coupled to and provide analytics and other data to the inventory management and analytics component. Inventory management systems are well-known in the art and are not described in detail here.

As another example, the electronic healthcare platform may include a traffic analytics component, such as, for example, Google Analytics®. The content aggregators 218 may aggregate traffic analytics data into the record database 212, such as data relating to number of users of the various front-end user interfaces, time spent using the various interfaces, click-through rates, etc. Traffic analytics systems are well-known in the art and are not described in detail here.

The electronic healthcare platform may further include one or more fulfillment partners, which fulfill orders for products. The interface to the fulfillment partners may be managed in the back-end system 224.

As another example, the electronic healthcare platform may include a recommendation engine. The recommendation may provide a personalized set of products and/or recommendations that are directly relevant to customers. The recommendations may be based on a number of factors, including input from healthcare professionals, a patient's medical codes and history, a patient's personal information (such as, for example, location, age, sex, income, health insurance coverage, purchasing trends, etc.). The recommendations may further be based on reviews and/or other feedback provided by other users of the electronic healthcare platform or third-party reviews of the products.

As another example, the electronic healthcare platform may further include a customer relationship management (CRM) component, such as, for example, Salesforce®. The front-end interface(s) may also be communicatively coupled to and provide analytics and other customer information to the CRM component.

As another example, the electronic healthcare platform may further include a call center. The call center component may include call, self-service, chat (web, video), and automated (bot) components. The front-end system 220 may be communicatively coupled to the call center component and provide call, chat, agent interface, and analytics to the call center component.

As another example, the electronic healthcare platform may include a personalization engine. The personalization engine may provide a personalized set of information and/or recommendations that are directly relevant to a customer. The personalized resources may be provided to a user based on known information about the user as well as analytics based on the information known about the user.

The content aggregators 218 may further import electronic health records (EHR) of patients into the record database 212. This may occur as part of a patient-discharge workflow when a patient is discharged from the doctor's office, hospital, or other medical facility. These EHR and related notes may be imported either automatically through a healthcare provider's HER system or manually input into a patient's profile within the platform (e.g., the patient's Care Plan) so that the discharge instructions may be reviewed by the patient later and additional information and/or relevant products/services may be found. In addition or in the alternative, EHR may be added to the record database 212 (minus any identifying information) to add to the number of data points within the database to allow for more accurate and/or thorough analysis using machine learning and/or artificial intelligence.

The electronic healthcare platform may include the ability to perform analytics on the data in the record database. The analytics performed may include, for example, predictive analytics and prescriptive analytics. Predictive analytics are used to determine what may happen in the future, based on past results. Prescriptive analytics are used to affect future results by modifying current information and behavior.

The code-lock system 216 is communicatively coupled to the record database 212, such that it can read and write to the record database 212. The code-lock system 216 performs analytics on the data in the record database and comprises a suite of modules for adding, maintaining, or removing concept associations. The code-lock system 216 may use, for example, expert annotation (e.g., manual coding), human-created heuristics, and the application of machine learning. The code-lock system 216 is loosely coupled with the platform and with other modules within the code-lock system 216, such that updates to the code-lock system 216 have minimal impact on the electronic healthcare platform.

The code-lock system 216 is configured to observe, identify, and/or create increasingly complex relationships and/or increasingly complex patterns within the data stored in the database. For example, the code-lock system 216 may use artificial intelligence and/or machine learning to identify relationships within the record database 212, especially between concepts and medical codes. As explained above, the electronic healthcare platform may include multiple patient profiles, each of which may have one or more medical codes associated them. As a result, the code-lock system 216 may be able to use artificial intelligence and/or machine learning to identify correlations between medical codes, on the one hand, and other medical codes and/or patient characteristics (e.g., age, height, weight, sex, medical history, etc.). Based on these identified correlations in the data, the code-lock system 216 may create additional cross-references and/or links associated with the data.

The code-lock system 216 may analyze data contained in the record database 212 to identify patterns and/or trends. For example, the code-lock system 216 may analyze data contained in the record database 212 to identify trends, such as regional preferences for products and/or services, socioeconomic preferences for products and/or services, etc. As another example, the code-lock system 216 may have access to purchasing information stored in the record database 212 (e.g., via data imported through content aggregators 218), so it may be able to determine which products are repeatedly purchased by which patients, and what medical codes are associated with those patients and/or products. From that information, the code-lock system 216 may determine that certain products are likely to be more effective for a patient with a particular associated medical code. It may further associate various keyword searches run by multiple users (e.g., via auxiliary services 214) with the end result of their visit (e.g., purchase vs. no purchase, articles reviewed, etc.) to the electronic healthcare platform and compute a weighted value of how relevant certain products and/or services are to certain keyword searches.

The code-lock system 216 may perform the functions of curated data import, medical coding, text analysis, classification, and performing heuristics.

The code-lock system 216 performs curated data import by configuring desired associations between concepts based on associations prepared by a person (e.g., a partner content provider, an account manager, an administrator, a medical expert, etc.).

The code-lock system 216 performs medical coding by explicitly associating a concept with a medical code performed by an expert (e.g., medical expert, doctor, healthcare professional).

The code-lock system 216 performs heuristics by using human-identified rules for associating concepts in the platform. This may be accomplished by using a concept query API to identify concepts to be associated. For example, the code-lock system 216 may use a human-identified rule to associate medical code E11.9 with all concepts from the Healthwise source that are articles and contain the words “diabetes” and/or “insulin.”

The code-lock system 216 may use machine learning to associate concepts within the platform. The code-lock system 216 may include one or more machine-learning modules that define a projection to map platform content data into a format suitable for automated learning. The modules apply the defined projection to the platform's content database, apply a machine-learning technique to the content, and propose associations to add to the content or remove from the content based on the results of the machine learning. The proposed changes are grouped into a batch. Each association in a batch includes provenance information, including metadata about this process, including but not limited to an identifier of the machine-learning module, and a time the batch was created.

The code-lock system 216 machine-learning modules may include, for example, a text analysis module and a concept categorization and classification module.

The text analysis module applies commonly available tools for measuring similarity of text in content. For example, for a “new concept,” the text analysis module finds “existing concepts” (i.e., concepts that have already been code-locked) with similar text to the new concept. The text analysis module identifies all the concepts related to the existing concepts, which are referred to as “related concepts.” The text analysis module proposes associations for the “new concept” with all “related concepts.” The concept categorization and classification module applies commonly available tools for performing automated clustering. The concepts within the platform can be organized into a concept graph. The concept graph can be partitioned (i.e., “clustered”) based on features of the concepts (i.e., “nodes”) and their associations (i.e., “edges”). The features of concepts in the clusters are used to define classes. Classification models of the module are then trained to place “new concepts” into the clusters. The concept categorization and classification module proposes associations for the “new concept” based on the classification.

The machine-learning modules of the code-lock system 216 may use metadata about concepts and associations during the code-lock process. A machine-learning module can be trained to take into account the provenance of existing associations, e.g., some associations may be assigned different weights during partitioning based on what other machine-learning module(s) created the association, when the association was created, or if a human has explicitly confirmed the association.

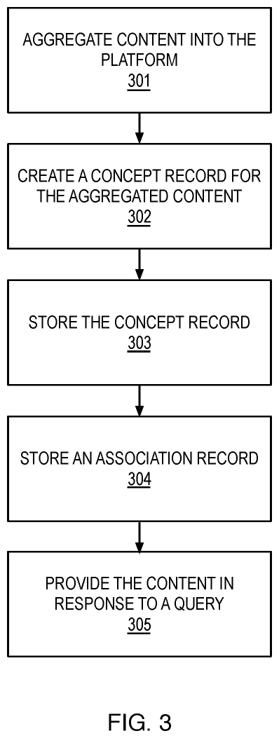

FIG. 3 depicts an exemplary method of providing content to a user of the electronic healthcare platform.

Referring to FIG. 3, at step 301, content is aggregated into the electronic healthcare platform by storing the content in a record database. As explained above, the content that is aggregated into the electronic healthcare platform may include medical codes from one or more medical coding systems (e.g., ICD-9 and ICD-10 codes, SNOMED CT codes, HSPC codes, NUCC medical codes, CPT codes). The content that is aggregated may further include licensed content from third parties (e.g., articles), original content (e.g., articles, questionnaires), product content (e.g., medical products for purchase, such as over-the-counter drugs, bandages, etc.), and service content (e.g., medical services, such as healthcare providers and/or in-home health care services).

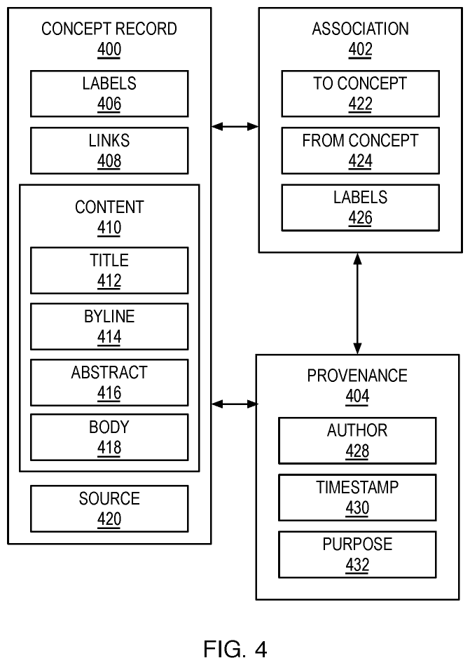

At step 302, a concept record is created for the aggregated content. Each piece of content that is aggregated into the platform may be associated with one or more concept records. As discussed in the context of FIG. 4, a concept record includes metadata relating to the content, such as labels relevant to the content and links/references to the content at the original content source. The labels identify concepts (e.g., by a concept ID) to which the content is relevant and may be created based on one or more medical codes that are associated with the piece of content. For example, one of the licensed content sources may include an informational article about type 2 diabetes. When the concept record for that article is created, the platform may label the article with “diabetes,” “insulin,” and “type 2,” “hyperglycemia,” and “insulin resistance,” for example. It may further associate the article one or more medical codes that are relevant to type 2 diabetes, such as E11.9.

At step 303, the concept record is stored in the record database. The concept record is stored in the record database such that it is linked in the record database to an association record and a provenance record (discussed in more detail in the context of FIG. 4). The association record links the concept record to other related concept records.

At step 304, an association record is stored in the record database. The association record may link the concept record with one or more other concept records in the record database. The association record may be created based on information contained in the concept record. The association record may be created based on information contained in the original content.

At step 305, content is provided in response to a query. The content that is provided is determined to be relevant to the query based on the concept associated with the content, the concept record stored in the record database, the association record related to the content, or any combination of the preceding. For example, in the case of the informational article about type 2 diabetes, an embedded component may display that article on a third-party partner website (which may not be same website where the article is originally located). The article that is displayed on the third-party partner website is determined based on one or more concept records associated with the article through a concept or an association record (e.g., based on a similar or related medical code). For example, if the partner website that the article is displayed on through the embedded component may be related to type 2 diabetes, so the platform generates the association between the content on the website and the content being displayed in the embedded component on the website.

The method described in FIG. 3 may be implemented on one or more systems configured as shown in FIG. 1 and FIG. 2. For example, the method described in FIG. 3 may be performed by a back-end server comprising a memory and a processor, with the processor being configured to perform the method described in the context of FIG. 3.

FIG. 4 depicts an exemplary structure of a concept record stored in the record database of the electronic healthcare platform.

As explained above, the electronic healthcare platform maintains multiple concept records related to the aggregated content in the platform. The multiple concept records are linked by one or more associations. Referring to FIG. 4, concept record 400 is stored in the record database with at least one association 402 and at least one provenance 404.

The concept record 400 may include semi-structured data to represent a concept. The concept record 400 includes one or more labels 406 and links 408. The labels and links contain information about the content 410 to which the concept is related. The concept record 400 further includes metadata information about the content 410 to which it is related, such as the title 412, byline 414, abstract 416, body 418, and source 420. This information and metadata in the concept record 400 allows the source content to be accessed as well as searched and analyzed for one or more associations.

As mentioned, the concept record may include metadata, which is information used by the platform to manage and maintain concept records, e.g., concept type, concept importance, etc. The concept record includes concept information that is normalized for use by the platform, e.g., for client visualization or search. The concept source includes detailed information about how to access the original concept information from the source.

The association 402 represents a relationship between concepts, e.g., the relationship between a health condition and a medical code or between a product and a health condition. One or more associations 402 are determined by the code-lock system of the platform to link one or more concepts with one or more other related concepts. Each association 402 includes a “to concept” field 422 and “from concept” field 424, which are used to link two or more concepts to one another. The association 402 may further include one or more labels 426, which further allows the association 402 to contain relevant details for the association.

The provenance 404 includes detailed information about a record (e.g., a concept record 400 or an association 402). The detailed information included in the provenance 404 may include, for example, the author 428 of the concept, a timestamp 430 of the concept (e.g., when the concept record 400 was created), the purpose 432 for which the concept record 400 was created, the method by which the record was created (e g, manually, using machine-learning), any approvals or restrictions on the use of the record, etc. As can be seen from FIG. 4, both concepts and associations may each include a provenance record.

In one embodiment, the concept records 400 are configured such that they comply with REST architectural principles and/or HTTP protocols and semantics. Delivery of the concept records 400 may be scaled by use of content-delivery networks (CDNs). In addition, the clients (e.g., web sites, apps, etc. shown in FIG. 1 and FIG. 2) may cache concept records for local use to make them more efficient.

The concept records 400 may be queried using various known database queries. For example, the concept records may be queried to return all content associated with a particular concept, or to return the source content associated with a particular concept, or to return all content that meets specific criteria, such as all articles that were published after a particular date. In addition, the concept records include graph query support (e.g., Cypher, SPARQL, GraphQL), which allows for return of all articles associated with one or more medical codes from a set of medical codes that are associated with a particular concept that were added by “user A” after a particular date.”

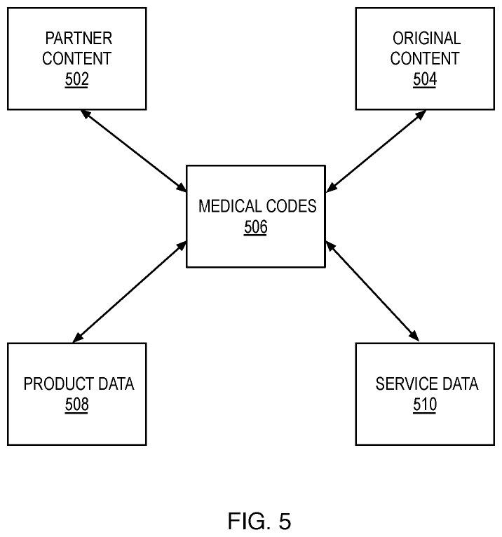

FIG. 5 depicts a relationship between different types of content and/or concepts within the platform and medical codes used within the platform.

Referring to FIG. 5, the electronic healthcare platform includes various types of content, such as partner content 502, original content 504, product data 508, and service data 510. The electronic healthcare platform further includes medical codes 506, and each of the partner content 502, original content 504, product data 508, and service data 510 are associated with one or more of the medical codes 506. The product data 508 is further associated with samples and e-commerce components within the system. The service data 510 is further associated with providers and services within the system.

As explained above in the context of FIG. 2, the platform analyzes source content as comprising concepts and associations between concepts. Content within the platform may be aggregated from multiple content sources, such as, for example, partner content 502 (e.g., licensed content from third-parties such as Healthwise), public standards and/or conventions (e.g., medical codes 506), original content 504 authored and/or owned by the platform, original content 504 authored by independent sources (e.g., bloggers, etc.) owned and/or curated by the platform. Each content source includes a model or representation of concepts and a model or representation of associations. The platform defines a model of concepts and associations that augments the original source content.

When a new content source is added to the platform (e.g., ingested, linked, etc.), the platform aggregates the new content source into the aggregated content. In one embodiment, the platform may be configured to maintain the original content from the added content source maintained while also remaining robust to any changes in original source content (e.g., by creating a copy of the ingested/linked content). The platform may be configured to incorporate human feedback about the content as the new content source is added (e.g., false negatives, false positives) or machine-learned feedback either immediately (or in near real-time) or at a later time.

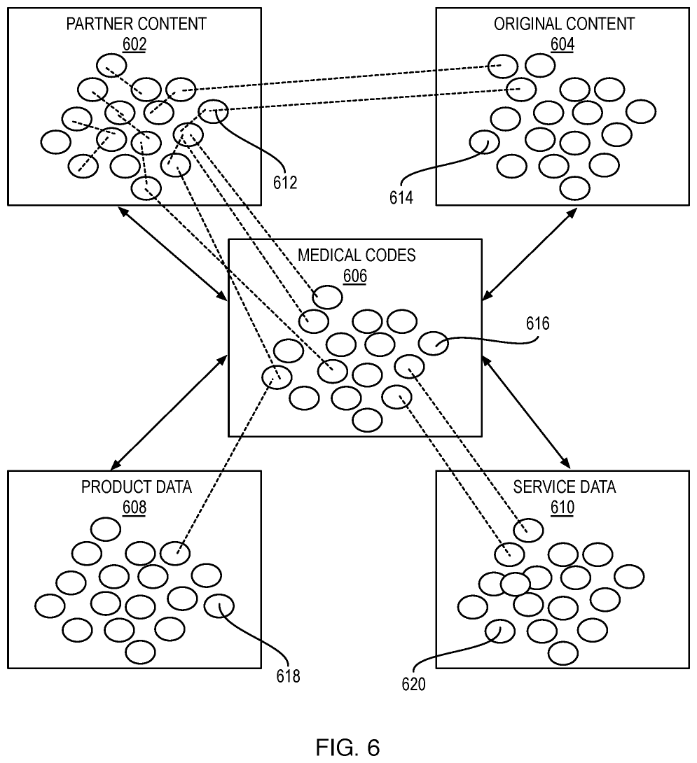

FIG. 6 depicts associations between various types of source content within the platform.

Referring to FIG. 6, each of the content bubbles 612, 614, 616, 618, and 620 shown in FIG. 6 represents a piece of content (e.g., partner content 602, original content 604, medical codes 606, product data 608, and service data 610) within the platform. The content within the platform may already include its own associations between data and/or concepts. For example, an article on a particular medical condition (e.g., from partner content 602) may already contain links to other related articles (e.g., from original content 604), identification and descriptions of the various relevant medical codes related to the condition (e.g., from medical codes 606), and/or links to products for treating that condition (e.g., product data 608). These pre-existing associations in content are represented by the small-dashed lines shown in FIG. 6.

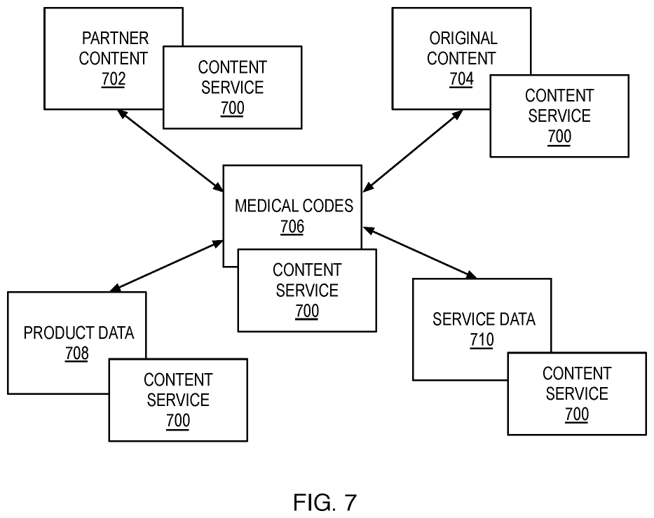

As explained above, the code-lock system (e.g., element 216 in FIG. 2) within the platform may identify the existing associations and/or generate additional associations between the concepts. The platform's content service defines a model of concepts and associations that augment the original source content, as shown in FIG. 7. The code-lock system uses one or more algorithms for constructing and maintaining associations between concepts in the platforms. The associations may be associations between concepts from different sources (e.g., associations between a health-related concept and a product concept) or associations between concepts in the same source that did not exist before (e.g., associations between one product concept to a “related” product concept).

FIG. 7 depicts associations between various types of source content within the platform along with the platform's content service providing an added model of concepts and associations that augment the original source content.

Referring to FIG. 7, the content sources (e.g., partner content 702, original content 704, product data 708, service data 710, and medical codes 706) from FIG. 5 are shown; however, in FIG. 7, these content sources further include instances of the platform's content service 700, which augment the original source content.

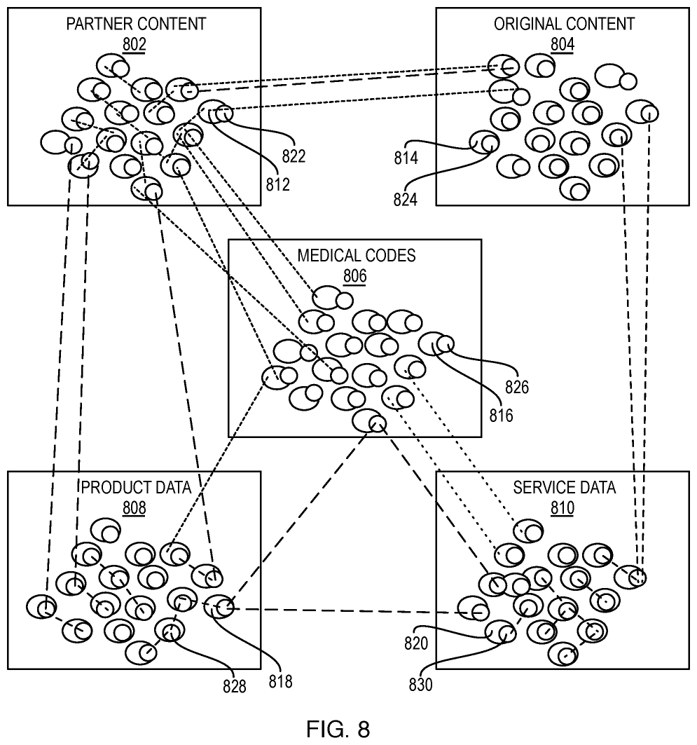

FIG. 8 depicts associations between various types of source content within the platform as well as the platform's content service augmenting the source content with concepts and associations within the platform.

Referring to FIG. 8, the platform's content service analyzes the source content to determine concepts and associations for each of the pieces of source content. The source content is shown as bubbles 812, 814, 816, 818, and 820, the concepts from the content service are shown as bubbles 822, 824, 826, 828, and 830 and the associations between the concepts are shown with a dashed line between the content and concepts in FIG. 8. The platform maps the original source content into the platform's content model. By mapping the original source content into the platform's content model, the platform may define new and/or additional associations between concepts in the content that do not already exist in the original source content. These new/additional associations in content are represented by the large-dashed lines shown in FIG. 8 (overlaid on the small-dashed lines from FIG. 6).

As discussed above, the electronic healthcare platform includes a CarePlanner functionality that user of the platform can access through the platform's user-interface layer. The CarePlanner functionality may be provided as a website that allows users to access the electronic healthcare platform via a web browser or a mobile app that allows users to access the electronic healthcare platform via a mobile device, such as a smartphone or tablet. The CarePlanner functionality provides information on health conditions, treatments, tests, wellness, diet, nutrition, and other health-related content from a variety of sources. The CarePlanner functionality uses the electronic healthcare platform's code-lock system (described above, for example, in the context of FIG. 2) to provide recommendations for articles, products, and/or services that are relevant to specific health conditions based on medical codes and associated concepts. The CarePlanner functionality integrates with the platform's content service (described above in the context of FIG. 2) and the platform's web component SDK (described above in the context of FIG. 2).

Users of the electronic healthcare platform may have a personalized Care Plan stored in the platform, which is a profile tailored to the user that contains health-related information specific to the user. A user's Care Plan is private to the user, but the user may also give access to their Care Plan to one or more of their healthcare professionals (e.g., doctors, nurses, home health care service providers, caregivers, etc.). A user's Care Plan may include information relating to that user's medical conditions, ailments, symptoms, and/or treatments.

A Care Plan may be created and/or developed by the user on their own, for example, as they research relevant medical conditions/ailments themselves before seeing a healthcare professional, or it may be created and/or developed by the user's healthcare professional, for example, during the course of treatment of the user by the healthcare professional. A user's Care Plan may include one or more medical codes associated with the user. The medical codes may be added by the user or the user's healthcare professional. In some embodiments, the medical codes may be gathered from the user's EHR through an integrated EHR system.

The electronic healthcare platform may allow for partnerships with one or more medical products companies. Through such partnership, the platform may provide sample products free of charge to patients and other users. For example, a user may search by the medical code of their medical condition, and the search may return a list of suggested products for that medical condition along with an option to electronically request a free sample of one or more of the suggested products.

In addition, the electronic healthcare platform may be integrated into other healthcare services and/or online platforms (such as EHR systems), so that when a patient is released from a hospital, doctor's office, or other medical facility, the patient's recommended services and products may be accessible within the platform, which will help the patient meet their health goals.

Within the Care Plan, a healthcare professional may access a user's Care Plan (with the user's permission), and review content (e.g., articles, products, and services) within the platform to suggest/recommend to the user by adding the content to the user's Care Plan. Similarly, the user may review their own Care Plan and add content to the Care Plan.

A user's Care Plan may include, for example, Condition Summary, My Products, My Services, and Recommended Articles information.

The Condition Summary within a user's Care Plan may include Doctor's Notes and My Conditions. The Doctor's Notes information may include recommendations for articles to review, treatments to pursue, and/or products to use made by one or more of the user's healthcare professionals. The My Conditions information may include a list of medical conditions, ailments, and/or symptoms associated with the user. The conditions/ailments/symptoms for a user may be entered by the user or by one or more of the user's healthcare professionals. The conditions may be selected from an existing list of conditions provided by the front-end user interface, or the user may type their condition. The electronic healthcare platform may automatically associate the entered conditions/notes with medical codes and/or keywords in the back-end database (via the content service). The front-platform may provide suggestions to the user based on the entered conditions/notes and/or any medical codes associated with the user.

The My Products information may include a list of products selected by the user, suggested by the platform (via the content services) based on code-locked concepts and/or associations, and/or recommended to the user by one or more healthcare professionals. The front-end user interface may include the option to purchase and/or request free samples of one or more of the products in the list of products using the electronic healthcare platform's e-commerce component.

The My Services information may include a list of services and/or service providers selected by the user, suggested by the platform (via the content services) based on code-locked concepts and/or associations or location of the user and the services, and/or recommended to the user by one or more healthcare professionals. The front-end user interface may include the option to contact one or more of the service providers in the list of providers.

The Recommended Articles information within a user's Care Plan may include a list of links to articles within the platform selected by the user, suggested by the platform (via the content services) based on code-locked concepts and/or associations, and/or recommended to the user by one or more healthcare professionals.

The electronic healthcare platform may further include additional dimensions of information, such as, for example, financial information, legal information, and residential information. Financial information may include information relating to financial planning and planning for medical and other related expenses. Legal information may include information relating to estate planning, succession planning, wills and estates, DNR directives, etc. Residential information may include information relating to logistics of in-home care, home modifications, live-in care providers, assisted living homes, etc.

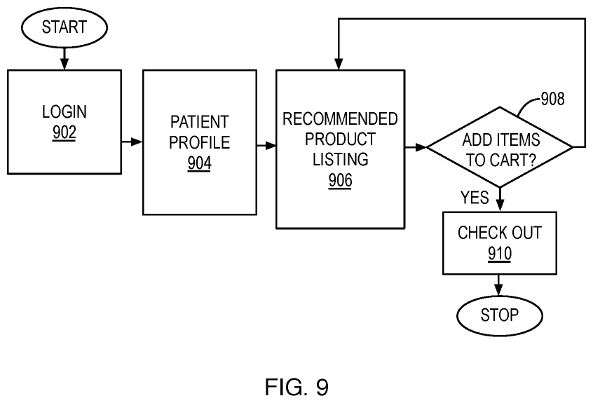

FIG. 9 depicts an exemplary process flow for a user of the electronic healthcare platform to purchase a recommended product.

Referring to FIG. 9, the user may log in to their account on the electronic healthcare platform, at step 902. Once logged in, the user may access their Patient Profile within the electronic healthcare platform, at step 904. At step 906, the user may view their Recommended Product Listing, which is a list of products that have been selected by the user, suggested by the platform (via the content service) based on one or more concepts and/or associations, or recommended to the user by their healthcare professional, as described in the context of a Care Plan above. The user may view information relating to the product, such as product details, specifications, reviews, etc. The user may either add some or all of the recommended items to their cart (step 908). Once the user has completed shopping, they may check out and order the products they have selected, at step 910.

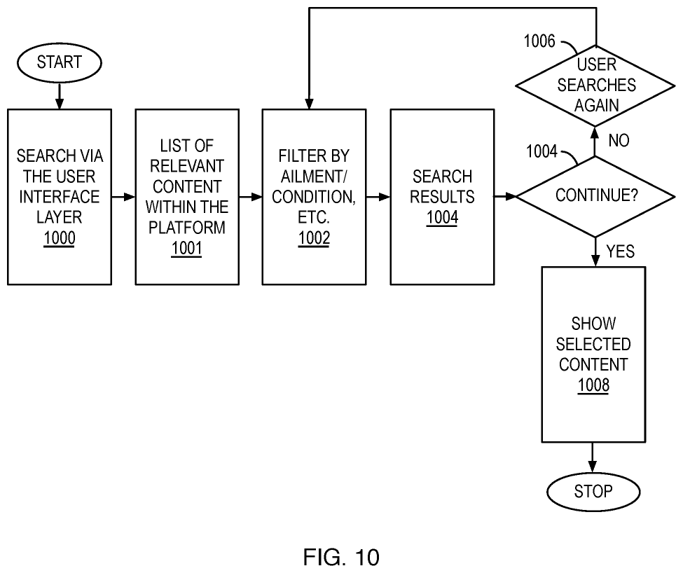

FIG. 10 depicts an exemplary process flow for finding relevant medical content within the electronic healthcare platform.

A user of the electronic healthcare platform may want to research or find content relevant to a particular medical issue or condition within the platform. For example, if the user of the platform is a patient, they may want to read articles about their issue/condition, find products specific to their issue/condition, or find a nearby healthcare provider. If the user of the platform is a caregiver, they may want to find articles relevant to the issues/conditions of the person in their care to assess if that person's symptoms are normal before contacting that person's doctor. If the user of the platform is a healthcare professional, they may want to suggest specific articles and/or products/services to their patient so that the patient can review the articles and/or order products or hire a service provider that is right for them.

For example, a user may want to learn more about type 2 diabetes and how to manage it. Or a healthcare professional may want to recommend incontinence products specific to their patient's needs and add those recommended products to their patient's Care Plan. The healthcare professional may also want to recommend mobility products appropriate for someone of the patient's height and weight. In these situations, both the healthcare professional and the patient may be a user of the electronic healthcare platform.

As shown FIG. 10, the user may begin with a search for information and/or products/services related to a specific topic via the user interface layer, at step 1000. For example, the user may want to learn about type 2 diabetes as well as to either purchase or request a sample of personal care products relating to diabetes (e.g., over-the-counter medicine, vitamins, bandages, therapy products, etc.). The search may begin at a third-party website, such as Google's search engine, a platform partner portal such as a healthcare provider's EHR system's patient portal, or a web-based or mobile application associated with the platform. As discussed in the context of FIG. 1, the user-interface layer of the platform includes web-based applications or websites, as well as partner portals that allow a user to access the platform's content from outside of the platform. At step 1001, the user is presented with search results that provide a list of relevant content (e.g., medical codes, articles, products, etc.) from the platform's content. In the example of the user searching for content relating to type 2 diabetes, the relevant content is determined by the platform's content service based on one or more concepts and/or associations related to the user's search for type 2 diabetes. At step 1002, the user may filter the results of the search using additional narrowing criteria, such as ailment, condition, symptoms, etc. The filter options may be based on the concept associations. For example, the filter may show additional relevant topics identified using associated concepts. The user may select to include one or more of the additional identified topics in the search results. At step 1003, the user is presented with a filtered list of the search results and may review a listing of the relevant content based on the filter parameters. In the example being used here, the search results may include articles about type 2 diabetes, articles about how to manage type 2 diabetes based on lifestyle, recommended blood sugar monitors, recommended nearby doctors who specialize in diabetes care, and articles relating to insulin resistance and/or other conditions often associated with type 2 diabetes. At step 1004, the user may continue to view one or more pieces of specific content returned as a search result. If the user wants to update their search to broaden/narrow the results, they can do so at step 1006. When viewing specific content from the search results, at step 1008, the user may review the selected content. The selected content also includes related content that is associated with the selected content through the platform's content services (e.g., related articles, products, and nearby services/service providers).

The electronic healthcare platform described herein may be implemented on one or more computers or computer systems within which a set of instructions, for causing the computer to perform any one or more of the functionalities discussed herein may be executed. The computer may operate as a standalone device or may be connected (e.g., networked) to other machines. In a networked deployment, the computer may operate in the capacity of a server or a client computer in a client-server network environment, or as a peer computer in a peer-to-peer (or distributed) network environment. The computer may be a server computer, a client computer, a personal computer (PC), a user device, a tablet PC, a laptop computer, a set-top box (STB), a personal digital assistant (PDA), a cellular telephone, an iPhone, an iPad, a Blackberry, a processor, a telephone, a web appliance, a network router, switch or bridge, a console, a hand-held console, a (hand-held) gaming device, a music player, any portable, mobile, hand-held device, or any machine capable of executing a set of instructions (sequential or otherwise) that specify actions to be taken by that machine.

The one or more computers used to implement the electronic healthcare platform described herein may include one or more processors, one or more memories (including a main memory and/or a non-volatile memory), one or more storage drives (including a machine-readable storage medium for storing, encoding, or carrying a set of instructions for execution by the computer and that cause the computer to perform any one or more of the methodologies of the presently disclosed technique and innovation.), one or more network interfaces, one or more displays, and one or more input devices (including a keyboard, mouse, touch-screen, etc.).

The electronic healthcare platform disclosed herein may be implemented on purpose-built devices such as a custom-built device with assembled hardware or the like. Additionally, the electronic healthcare platform disclosed herein could be implemented on existing RF communications devices that utilize communication modules embodying Wi-Fi®, Bluetooth®, Bluetooth® Low Energy, cellular long-term evolution (LTE®), or many other communications systems and devices.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EEPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including object oriented and/or procedural programming languages. Programming languages may include, but are not limited to: Ruby®, JavaScript®, Java®, Python®, PHP, C, C++, C#, Objective-C®, Go®, Scala®, Swift®, Rodin®, (Wand®, or the like. The program code may execute entirely on the user's computer, partly on the user's computer, as a stand-alone software package, partly on the user's computer, and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of providing personalized healthcare content through an electronic healthcare platform, the method comprising:
aggregating healthcare content by analyzing the healthcare content to determine a concept relevant to the healthcare content,
wherein the healthcare content includes medical code data, and
wherein the healthcare content further includes at least one content type selected from the group consisting of licensed content, original content, product content, and service content;
creating a concept record associated with the healthcare content based on the analysis of the healthcare content,
wherein the concept record includes concept metadata, and
wherein the concept record includes a reference to the healthcare content;
storing the concept record in a record database;
storing an association record in the record database that links the concept record with another concept record stored in the record database,
wherein the association record is created based on information contained in the concept record or information contained in the healthcare content; and
providing the healthcare content in response to a query, wherein the healthcare content is determined to be relevant to the query based on at least one of the concept record and the association record.

2. The method of claim 1, wherein the association record provides an association between a first concept and a second concept.

3. The method of claim 2, wherein the association between the first concept and the second concept is determined based on one or more medical codes related to the first concept and one or more medical codes related to the second concept.

4. The method of claim 1, wherein the concept relevant to the healthcare content is determined based one or more medical codes.

5. The method of claim 1, wherein the query is received from a front-end interface that includes a branded website that provides access to the aggregated content in the electronic healthcare platform.

6. The method of claim 1, wherein the query is received from a website that contains an embedded object from the electronic healthcare platform, the website being a third-party website that is not part of the electronic healthcare platform.

7. The method of claim 1, wherein a concept associated with search parameters of the query is used to determine whether the healthcare content is relevant to the query.

8. The method of claim 1, wherein the analyzing the healthcare content to determine a concept relevant to the healthcare content is performed using a machine-learning technique.

9. The method of claim 8, wherein the machine-learning technique determines that the concept is relevant to the healthcare content by performing a text-analysis technique to find similarities in the healthcare content.

10. The method of claim 8, wherein the machine-learning technique determines that the concept is relevant to the healthcare content by performing concept categorization and classification using a clustering technique to find similarities in the healthcare content.

11. A method of providing a personalized healthcare website as part of an electronic healthcare platform, the method comprising:
storing a profile associated with a user of the electronic healthcare platform, wherein the profile includes a medical code associated with the user; and
providing a recommendation to the user based on the medical code associated with the user,
wherein the recommendation includes a product associated with the medical code.

12. The method of claim 11, wherein the product is associated with the medical code using machine-learning.

13. The method of claim 11, wherein the recommendation further includes an article associated with the medical code.

14. The method of claim 11, wherein the recommendation further includes a healthcare service associated with the medical code.

15. An electronic healthcare platform, comprising:
a user interface layer, wherein the user interface layer is configured to display content using an embedded object;
a services layer, wherein the services layer includes an API configured to provide healthcare content to the embedded object; and
a content layer, wherein the content layer includes a content library containing healthcare content;
wherein the electronic healthcare platform associates healthcare content in the content library with a healthcare concept based on a medical code.

16. The electronic healthcare platform of claim 15, wherein the electronic healthcare platform further associates healthcare content in the content library with the healthcare concept using a medical condition.

17. The electronic healthcare platform of claim 15, wherein information relating to the healthcare concept is stored as a concept record in a record database.

18. The electronic healthcare platform of claim 15, wherein the concept record is linked to a related concept record in the record database using an association record stored in the record database.

19. The electronic healthcare platform of claim 15, wherein the embedded object is an HTML object associated with the healthcare concept using a concept ID.

20. The electronic healthcare platform of claim 15, wherein the content layer aggregates healthcare content from a content source by storing a reference to the content source in the record database.
